(19) United States
(12) Patent Application Publication
(10) Pub. No.: US 2012/0190691 A1
(43) Pub. Date: Jul. 26, 2012

(54) PHARMACEUTICAL COMPOSITIONS FOR TREATMENT OF RESPIRATORY AND INFLAMMATORY DISEASES

(75) Inventors: Thierry Bouyssou, Warthausen (DE); Abhya Gupta, Biberach an der Riss (DE); Peter Seither, Biberach an der Riss (DE)

(73) Assignee: BOEHRINGER INGELHEIM INTERNATIONAL GMBH, Ingelheim am Rhein (DE)

(21) Appl. No.: 13/191,500

(22) Filed: Jul. 27, 2011

(30) Foreign Application Priority Data
Jul. 28, 2010 (EP) 10171036.6

(51) Int. Cl. A61K 31/505; A61P 11/00; A61P 29/00; A61P 11/02; A61P 37/08; A61P 11/08; A61P 27/02; A61K 31/495; A61P 11/06

(52) U.S. Cl. 514/255.04; 514/256

(57) ABSTRACT

The present invention relates to pharmaceutical composition comprising a CRTH2 antagonist and at least one histamine receptor antagonist, medicaments containing said pharmaceutical compositions, and the use of said pharmaceutical compositions for treating respiratory and inflammatory diseases and conditions.

The present invention relates to pharmaceutical composition comprising a CRTH2 antagonist and at least one histamine receptor antagonist, medicaments containing said pharmaceutical compositions, and the use of said pharmaceutical compositions for treating respiratory and inflammatory diseases and conditions.

WO 2004/096777 discloses the CRTH2 antagonist [4,6-bis(dimethylamino)-2-(4-(4-(trifluoromethyl)benzamido)-benzyl)pyrimidin-5-yl]acetic acid (hereinafter referred to as CRTH2 antagonist of formula (1)), optionally in the form of its solvates, hydrates or their addition salts with pharmacologically acceptable acids or bases.

WO 2008/156781 discloses specific salts of said CRTH2 antagonist as well as pharmaceutical compositions containing said salts.

It was an objective of the present invention to provide pharmaceutical compositions for the treatment of respiratory and inflammatory diseases and conditions having enhanced activity. Said pharmaceutical compositions should allow for treating respiratory and inflammatory diseases and conditions with a smaller amount of active compounds and/or should allow for treating respiratory and inflammatory diseases and conditions in a more efficient way, thereby minimizing or obviating possibly existing adverse effects generally linked to any kind of treatment with an active compound in high doses and/or for a longer period of time.

### DETAILED DESCRIPTION OF THE INVENTION

According to the present invention this objective is achieved by providing a pharmaceutical composition comprising a CRTH2 antagonist of formula (1) (also referred to as CRTH2 antagonist (1),

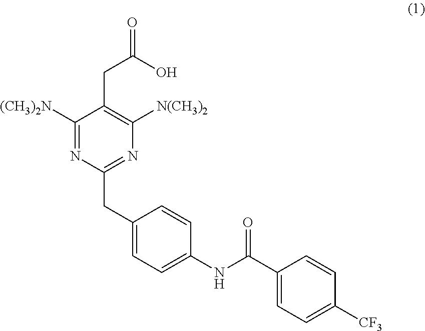

optionally in the form of its solvates, hydrates or salts with pharmacologically acceptable acids or bases, and at least one histamine receptor antagonist.

The pharmaceutical compositions according to the present invention show an activity which is significantly higher than the activity that would have been expected knowing the individual activities of each of the components. Thus, the pharmaceutical compositions should allow for treating respiratory and inflammatory diseases and conditions with a smaller amount of active compounds and/or should allow for treating respiratory and inflammatory diseases and conditions in a more efficient way.

Therefore, the present invention further relates to a pharmaceutical composition according to the invention for use in the treatment of respiratory and inflammatory diseases and conditions.

Another embodiment of the present invention relates to a method of treating respiratory and inflammatory diseases and conditions, comprising administering a therapeutically effective amount of a pharmaceutical composition according to the present invention to a patient in need thereof.

Another embodiment of the present invention relates to the use of a pharmaceutical composition according to the invention for making a medicament for treating respiratory and inflammatory diseases and conditions.

Yet another embodiment of the present invention relates to a unit-dosage form comprising the pharmaceutical composition according to invention.

In the pharmaceutical compositions according to the present invention the CRTH2 antagonists of formula (1) may be contained in a form selected from solvates, hydrates or salts with pharmacologically acceptable acids or bases.

Suitable salts have been disclosed in WO 2008/156781. For a review on additional suitable salts, see “Handbook of Pharmaceutical Salts: Properties, Selection, and Use” by Stahl and Wermuth, Wiley-VCH, 2002.

One embodiment of the present invention relates to pharmaceutical composition according to the invention, wherein the CRTH2 antagonists of formula (1) is present as a salt with a pharmacologically acceptable base, wherein the base is an amine selected from primary amines, including methylamine, ethylamine, ethanolamine, tris(hydroxymethyl)-aminomethane, and ethylenediamine; secondary amines, including dimethylamine, diethylamine, diisopropylamine, dibutylamine, di-sec-butylamine, dicyclohexylamine, diethanolamine, meglumine, pyrrolidine, piperidine, piperazine, and benzathine; tertiary amines, including trimethylamine, triethylamine, triethanolamine, and 1-(2-hydroxyethyl)-pyrrolidine; quaternary ammoniums, including choline, tetramethylammonium, and tetraethylammonium. More preferred are the pharmaceutical compositions according to the invention, wherein the amine is selected from ethylenediamine and choline. Particularly preferred are the pharmaceutical composition according to the invention, wherein the CRTH2 antagonist of formula (1) is present in the form of its choline salt.

The pharmaceutical compositions according to the present invention further contain the histamine receptor antagonist. Suitable histamine receptor antagonists preferably are selected from acrivastine, azatadine, azelastine, bamipine, brompheniramine, carbinoxamine, cetirizine, chlorphenoxamine, chlorphenaramine, clemastine, cexchlorpheniramine, cyproheptadine, desloratidine, dexbromphenarimine, dexchlorpheniramine, dimenhydrinate, dimetinden, diphenhydramine, doxylamine, ebastine, emedastine, epinastine, fexofenadine, hydroxyzine, ketotifen, levocetirizine, levocabastine, loratadine, meclozine, methdilazine, mizolastine, olopatadine, phenindamine, pheniramine, phenyltoloxamine, promethazine, pyrilamine, tecastemizole, trimepramine, trimethobenzamide, triprolidine, JNJ-7777120, PF-2988403 and CZC-13788, optionally in racemic form, as enantiomers, diastereomeres or as pharmacologically acceptable salts, solvates or hydrates.

More preferably the histamine receptor antagonist is selected from azelastine, cetirizine, desloratidine, ebastine, epinastine, fexofenadine, hydroxyzine, ketotifen, levocetirizine, loratadine, olopatadine and pyrilamine.

Particularly preferred are pharmaceutical compositions according to the present invention, wherein the histamine receptor antagonist is selected from cetirizine, desloratidine, fexofenadine and levocetirizine.

One particular embodiment of the present invention relates to pharmaceutical compositions according to the present invention, wherein the histamine receptor antagonist s cetirizine.

Another particular embodiment of the present invention relates to pharmaceutical compositions according to the present invention, wherein the histamine receptor antagonist is desloratidine.

Another particular embodiment of the present invention relates to pharmaceutical compositions according to the present invention, wherein the histamine receptor antagonist is fexofenadine.

Another particular embodiment of the present invention relates to pharmaceutical compositions according to the present invention, wherein the histamine receptor antagonist is levocetirizine.

### Pharmaceutical Compositions

The pharmaceutical compositions according to the present invention may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampoules, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The unit-dosage form comprising the pharmaceutical composition according to the present invention, usually comprises the CRTH2 antagonists of formula (1) in an amount of from 1 mg to 1000 mg, preferably in an amount of from 10 mg to 800 mg, more preferably in an amount of from 25 mg to 500 mg. Particularly preferred are unit-dosage forms according to the present invention comprising the CRTH2 antagonist of formula (1) in an amount of from 25 mg to 400 mg. All amounts of the CRTH2 antagonist of formula (1) given within the specification refer to the amount of the free compound of formula (1) irrespective of the particular form said antagonist is present in the pharmaceutical composition.

The unit-dosage form comprising the pharmaceutical composition according to the present invention usually comprise the histamine receptor antagonist in an amount from 0.1 mg to 1000 mg, preferably in an amount of from 0.5 to 500 mg, more preferably in an amount of from 1 to 200 mg. All amounts of the histamine receptor antagonist given within the specification refer to the amount of the free active compound irrespective of the particular form said antagonist is present in the pharmaceutical composition.

The unit-dosage form according to the present invention preferably comprises the CRTH2 antagonist of formula (1) in an amount of from 1 mg to 1000 mg and the histamine receptor antagonist in an amount of from 0.1 mg to 1000 mg.

In the unit-dosage forms comprising fexofenadine, said histamine receptor antagonist is usually present in an amount of from 1 to 500 mg, preferably in an amount of from 5 to 200 mg, and in particular in an amount of from 10 to 180 mg.

In the unit-dosage forms comprising desloratidine, said histamine receptor antagonist is usually present in an amount of from 0.2 to 200 mg, preferably of from 0.5 to 50 mg, and in particular of from 1 to 10 mg.

In the unit-dosage forms comprising cetirizine, said histamine receptor antagonist is usually present in an amount of from 0.2 to 200 mg, preferably of from 0.5 to 50 mg, and in particular of from 2.5 to 10 mg.

In the unit-dosage forms comprising levocetirizine, said histamine receptor antagonist is usually present in an amount of from 0.2 to 200 mg, preferably of from 0.5 to 50 mg, and in particular of from 1.25 to 10 mg.

The pharmaceutical composition according to the present invention, usually comprise the CRTH2 of formula (1) and the histamine receptor antagonist in a weight ratio of from 1:100 to 1000:1, preferably in a weight ratio of from 1:50 to 500:1, more preferably in a weight ratio of from 1:2 to 200:1, and in particularly in a weight ratio of from 1:1 to 100:1.

The pharmaceutical compositions according to the present invention may comprise the CRTH2 antagonist of formula (1), one or more additional CRTH2 antagonists which is/are different from the CRTH2 antagonist of formula (1), and more than one histamine receptor antagonist.

The pharmaceutical compositions according to the present invention may be administered alone, or in combination with one or more other ingredients.

Thus, the pharmaceutical compositions of the present invention may further comprise at least one active ingredient selected from the classes consisting of β2-adrenoceptor-agonists (short and long-acting beta mimetics), anti-cholinergics (short and long-acting), anti-inflammatory steroids (oral and topical corticosteroids), dissociated-glucocorticoidmimetics, PDE3 inhibitors, PDE4 inhibitors, PDE7 inhibitors, LTD4 antagonists, EGFR inhibitors, PAF antagonists, Lipoxin A4 derivatives, FPRL1 modulators, LTB4-receptor (BLT1, BLT2) antagonists, PI3-kinase inhibitors, inhibitors of non-receptor tyrosine kinases as for example LYN, LCK, SYK, ZAP-70, FYN, BTK or ITK, inhibitors of MAP kinases as for example p38, ERK1, ERK2, JNK1, JNK2, JNK3 or SAP, inhibitors of the NF-κB signalling pathway as for example IKK2 kinase inhibitors, iNOS inhibitors, MRP4 inhibitors, leukotriene biosynthese inhibitors as for example 5-Lipoxygenase (5-LO) inhibitors, cPLA2 inhibitors, Leukotriene A4 hydrolase inhibitors or FLAP inhibitors, non-steroidale anti-inflammatory agents (NSAIDs), DP1-receptor modulators, thromboxane receptor antagonists, CCR1 antagonists, CCR2 antagonists, CCR3 antagonists, CCR4 antagonists, CCR5 antagonists, CCR6 antagonists, CCR7 antagonists, CCR8 antagonists, CCR9 antagonists, CCR10 antagonists, CXCR1 antagonists, CXCR2 antagonists, CXCR3 antagonists, CXCR4 antagonists, CXCR5 antagonists, CXCR6 antagonists, CX3CR1 antagonists, Neurokinin (NK1, NK2) antagonists, sphingosine 1-phosphate receptor modulators, sphingosine 1 phosphate lyase inhibitors, adenosine receptor modulators as for example A2a-agonists, modulators of purinergic receptors as for example P2X7 inhibitors, histone deacetylase (HDAC) activators, bradykinin (BK1, BK2) antagonists, TACE inhibitors, PPAR gamma modulators, Rho-kinase inhibitors, interleukin 1-beta converting enzyme (ICE) inhibitors, Toll-like receptor (TLR) modulators, HMG-CoA reductase inhibitors, VLA-4 antagonists, ICAM-1 inhibitors, SHIP agonists, GABAa receptor antagonist, ENaC-inhibitors, Melanocortin receptor (MC1R, MC2R, MC3R, MC4R, MC5R) modulators, CGRP antagonists, Endothelin antagonists, Somatostatin agonists (SSTR1, SSTR2, SSTR3, SSTR4, SSTR5), TRP antagonists, in particular TRPV antagonists (TRPV1, TRPV2, TRPV3, TRPV4, TRPV5, TRPV6), TRPA antagonists, TRPC antagonists and TRPM antagonists, mucoregulators, immunotherapeutic agents, compounds against swelling of the airways, compounds against cough, CB2 agonists, retinoids, immunosuppressants, mast cell stabilizers, methylxanthine, opioid receptor agonists, laxatives, anti-foaming agents, antispasmodic agents, 5-HT4 agonists but also combinations of two or three further active substances.

The pharmaceutical composition according to the present invention additionally may contain one or more pharmaceutically acceptable carriers or excipients. The choice of excipient, to a large extent, depends on factors, such as the particular mode of administration, the effect of the excipient on the solubility and stability of the active ingredient, and the nature of the dosage form.

The pharmaceutical compositions according to the present invention may be formulated in various dosage forms for oral, parenteral, and topical administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art.

The pharmaceutical compositions according to the present invention may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions according to the present invention may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN®20), polyoxyethylene sorbitan monooleate 80 (TWEEN®80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

The pharmaceutical compositions according to the present invention may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions according to the present invention may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propylparabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. The capsules may also be coated as known by those of skill in the art in order to modify or sustain dissolution of the active ingredient.

The pharmaceutical compositions according to the present invention may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredients provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions according to the present invention for oral administration may be also provided in the forms of liposomes, micelles, microspheres, or nanosystems.

The pharmaceutical compositions according to the present invention may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions according to the present invention may be formulated as immediate or modified release dosage forms, including delayed-, sustained, pulsed-, controlled, targeted-, and programmed-release forms.

The pharmaceutical compositions according to the present invention may be co-formulated with other active ingredients which do not impair the desired therapeutic action.

### B. Parenteral Administration

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science.

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

The pharmaceutical compositions according to the present invention may be formulated for single or multiple dosage administration. The single dosage formulations are packaged in an ampule, a vial, or a syringe. The multiple dosage parenteral formulations must contain an antimicrobial agent at bacteriostatic or fungistatic concentrations.

The pharmaceutical compositions according to the present invention may be formulated as immediate or modified release dosage forms, including delayed-, sustained, pulsed-, controlled, targeted-, and programmed-release forms.

### C. Topical Administration

The pharmaceutical compositions according to the present invention may be administered topically to the skin, orifices, or mucosa. The topical administration, as used herein, include (intra)dermal, conjuctival, intracorneal, intraocular, ophthalmic, auricular, transdermal, nasal, vaginal, uretheral, respiratory, and rectal administration.

The pharmaceutical compositions according to the present invention may be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein may also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryopretectants, lyoprotectants, thickening agents, and inert gases.

The pharmaceutical compositions according to the present invention for topical administration may be formulated to be immediate release or modified release, including delayed-, sustained-, pulsed-, controlled-, targeted, and programmed release.

### D. Modified Release

The pharmaceutical compositions according to the present invention may be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile- or pulsed-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredients can also be modified by varying the particle sizes and polymorphorism of the active ingredients.

### Medical Indications

The pyrimidine derivative of the formula (1) shows excellent CRTH2 antagonistic activity. It is therefore, suitable especially for the prophylaxis and treatment of diseases associated with CRTH2 activity.

It has been found that the pharmaceutical compositions described herein have a beneficial effect in terms of bronchospasmolysis and reduction of inflammations in the airways, as well as inflammatory diseases of the joints, and allergic diseases of the oro-naso pharynx, skin or the eyes.

It has been found that this is particularly true for combinations of the CRTH2 compound of formula (1), with histamine receptor antagonists.

A further embodiment of the present invention relates to a method of treating an indication selected from respiratory diseases and conditions such as diseases of the airways and lungs which are accompanied by increased or altered production of mucus and/or inflammatory and/or obstructive diseases of the airways such as acute bronchitis, chronic bronchitis, chronic obstructive bronchitis (COPD), cough, pulmonary emphysema, allergic or non-allergic rhinitis or sinusitis, seasonal and perenial, chronic uritcaria, chronic sinusitis or rhinitis, nasal polyposis, chronic rhinosinusitis, acute rhinosinusitis, asthma, allergic bronchitis, alveolitis, Farmer's disease, hyperreactive airways, bronchitis or pneumonitis caused by infection, e.g. by bacteria or viruses or helminthes or fungi or protozoons or other pathogens, pediatric asthma, bronchiectasis, pulmonary fibrosis, adult respiratory distress syndrome, bronchial and pulmonary edema, bronchitis or pneumonitis or interstitial pneumonitis caused by different origins, e.g. aspiration, inhalation of toxic gases, vapors, bronchitis or pneumonitis or interstitial pneumonitis caused by heart failure, X-rays, radiation, chemotherapy, bronchitis or pneumonitis or interstitial pneumonitis associated with collagenosis, e.g. lupus erythematodes, systemic scleroderma, lung fibrosis, idiopathic pulmonary lung fibrosis (IPF), interstitial lung diseases or interstitial pneumonitis of different origin, including asbestosis, silicosis, M. Boeck or sarcoidosis, granulomatosis, cystic fibrosis, mucoviscidosis, or α1-antitrypsin deficiency;

or selected from inflammatory diseases and conditions such as inflammatory diseases of the gastrointestinal tract of various origins such as inflammatory pseudopolyps, Crohn's disease, ulcerative colitis, inflammatory diseases of the joints, such as rheumatoid arthritis, or allergic inflammatory diseases of the oro-nasopharynx, skin or the eyes, such as atopic dermatitis, seasonal and perenial, chronic uritcaria, hives of unknown cause and allergic conjunctivitis; and in particular selected from asthma, allergic and non-allergic rhinitis, allergic bronchitis, allergic conjunctivitis, farmers disease, nasal polyposis, chronic uritcaria, chronic sinusitis, atopic dermatitis, chronic rhinosinusitis and acute rhinosinusitis;
comprising administering a therapeutically effective amount of a pharmaceutical composition according to the present invention to a patient in need thereof.

A further embodiment of the present invention relates to the use of a pharmaceutical composition according to the present invention for making a medicament for treating respiratory and/or inflammatory diseases and conditions, particularly wherein the respiratory and/or inflammatory diseases or conditions are selected from asthma, allergic and non-allergic rhinitis, allergic bronchitis, allergic conjunctivitis, farmer's disease, nasal polyposis, chronic uritcaria, chronic sinusitis, atopic dermatitis, chronic rhinosinusitis and acute rhinosinusitis.

A further embodiment of the present invention relates to a pharmaceutical composition according to the present invention for use in the treatment of respiratory and inflamatory diseases and conditions, particularly wherein the respiratory and inflammatory diseases or conditions are selected from asthma, allergic and non-allergic rhinitis, allergic bronchitis, allergic conjunctivitis, farmers disease, nasal polyposis, chronic uritcaria, chronic sinusitis, atopic dermatitis, chronic rhinosinusitis and acute rhinosinusitis.

The present invention is now further illustrated by means of biological examples.

### BIOLOGICAL EXAMPLES

### A. Experimental Procedure

### Animals

Male and female Dunkin-Harley guinea pigs were obtained from the Experimental Animal Breeding Centre of Harlan Winkelmann (Germany). After fasting overnight, but with free access to drinking water, animals with body weight of 400-500 g were used.

### Instrumentation and Measurements

Bronchospasm was recorded with a modified version of the method of Konzett-Röβler (described by Walland et al, in “Compensation of muscarinic bronchial effects of talsaclidine by concomitant sympathetic activation in guinea pigs”, European Journal of Pharmacology, Volume 330, Issue 2-3, 9 Jul. 1997, Pages 213-219). The animals were ventilated by means of a piston pump (starling ventilator, Hugo Sachs Elektronik, Germany) at a stroke volume of 1 ml/100 g body weight and at a rate of 60 strokes per min. The tubing which connected the tracheal cannula with the ventilator was provided with a branch leading to the bronchospasm transducer (bronchospasm transducer 7020, Ugo Basile, Italy). The air flow measurement is based on the hot wire anemometer principle. Any air flow will reduce the temperature of the wire, thereby decreasing its resistance in proportion of the flow. As the wire element is one arm of a Weaststone bridge, variation in resistance generate a proportional voltage output which feeds the amplifier/recording system (Notocord-hem, Notocord, France). Blood pressure and heart rate are monitored from a carotid artery in order to check the anesthesia and the variability of the preparation.

### Experimental Protocol

Guinea pigs were sensitized on 2 consecutive days with 20 μg ovalbumin (OVA) (Sigma, St. Louis, Mo.) and 20 mg Al(OH)₃ in 0.5 ml saline, which were administered subcutaneously. Experiments, which included OVA challenge, were conducted 2 weeks after sensitization. Approximately 1 hour before OVA challenge, the animals were anaesthetized with an intraperitoneal injection of 50 mg/kg pentobarbital. Anesthesia was prolonged by intravenous infusion of pentobarbital (15 mg/kg/h) via the jugular vein. A tracheal cannula was introduced after tracheotomy for artificial ventilation. The internal jugular vein was cannulated for drug injection while the left carotid artery was cannulated for measuring blood pressure and heart rate. 30 minutes after the beginning of air flow and blood pressure measurements, bronchoconstriction is triggered by an OVA challenge given at a fixed dose of 50 μg/kg inhaled.

The test compounds or combinations of compounds were administered orally 2 hours before the OVA challenge.

The control group comprises 12 guinea-pigs, test compounds comprise 2 to 4 guinea-pigs.

At the end of the experiment, animals are euthanized by an overdose of pentobarbital (100 mg/kg i.v.).

OVA challenge in control guinea-pigs induces an overflow of 60 (±13) ml which is used as 100% bronchospasm. Bronchoprotection of the drug (i.e. inhibition of OVA broncho-constriction) is expressed as a percentage of inhibition of the increase in overflow induced by OVA in the control animals.

### B. Results

### 1. [4,6-bis(dimethylamino)-2-(4-(4-(trifluoromethyl)benzamido)benzyl)pyrimidin-5-yl]-acetic acid (CRTH2 antagonist (1)) (Reference Example)

| CRTH2 antagonist (1) | | |
|---|---|---|
| [mg/kg p.o.]¹⁾ | overflow [ml] | bronchoprotection [%] |
| 0.00 | 60 | 0% |
| 0.01 | 63 | n.d. |
| 0.03 | 65 | n.d. |
| 0.10 | 69 | n.d. |
| 0.30 | 60 | n.d. |
| 1.00 | 65 | n.d. |
| 3.00 | 61 | n.d. |
| 10.00 | 65 | n.d. |

¹⁾CRTH2 antagonist (1) was administered in the form of its ethylenediamine salt; the dose is calculated based on the free compound of formula (1);

As can be seen CRTH2 antagonist of formula (1) in this assay has no effect by itself on the ovalbumin-induced bronchospasm up to 10 mg/kg p.o.

### 2. CRTH2 Antagonist (1)+Pyrilamine

| Pyrilamine | CRTH2 antagonist (1) | overflow | bronchoprotection |
|---|---|---|---|
| [mg/kg p.o.]²⁾ | [mg/kg p.o.]¹⁾ | [ml] | [%] |
| 2.0 | 0.0 | 33 | 45 |
| 2.0 | 0.1 | 41 | 32 |
| 2.0 | 0.3 | 25 | 58 |
| 2.0 | 1.0 | 19 | 68 |
| 2.0 | 3.0 | 12 | 80 |
| 2.0 | 10.0 | 10 | 83 |

¹⁾CRTH2 antagonist of formula (1) was administered in the form of its ethylenediamine salt; the dose is calculated based on the free compound of formula (1);
²⁾Pyrilamine was administered in the form of its maleate salt; the dose is calculated based on free pyrilamine

### 3. CRTH2 Antagonist of Formula (1)+Fexofenadine

| Fexofenadine | CRTH2 antagonist (1) | overflow | bronchoprotection |
|---|---|---|---|
| [mg/kg p.o.]³⁾ | [mg/kg p.o.]¹⁾ | [ml] | [%] |
| 0.5 | 0.0 | 39 | 35 |
| 0.5 | 0.01 | 33 | 40 |
| 0.5 | 0.03 | 10 | 83 |
| 0.5 | 0.1 | 9 | 85 |
| 0.5 | 0.3 | 4 | 93 |
| 0.5 | 1.0 | 7 | 88 |
| 0.5 | 3.0 | 6 | 90 |
| 0.5 | 10.0 | 10 | 83 |

¹⁾CRTH2 antagonist of formula (1) was administered in the form of its ethylenediamine salt; the dose is calculated based on the free compound of formula (1);
³⁾Fexofenadine was administered in the form of its hydrochloride salt; the dose is calculated based on free fexofenadine

### 4. CRTH2 Antagonist of Formula (1)+Desloratidine

| Desloratidine | CRTH2 antagonist (1) | overflow | bronchoprotection |
|---|---|---|---|
| [mg/kg p.o.]⁴⁾ | [mg/kg p.o.]¹⁾ | [ml] | [%] |
| 2.0 | 0.0 | 34 | 43 |
| 2.0 | 0.1 | 24 | 60 |
| 2.0 | 0.3 | 22 | 63 |
| 2.0 | 1.0 | 20 | 67 |
| 2.0 | 3.0 | 14 | 77 |
| 2.0 | 10.0 | 18 | 70 |

¹⁾CRTH2 antagonist (1) was administered in the form of its ethylenediamine.salt; the dose is calculated based on the free compound of formula (1);
⁴⁾Desloratidine was administered in the form of its hydrochloride salt; the dose is calculated based on free desloratidine

### 5. CRTH2 Antagonist of Formula (1)+Cetrizine

| Cetrizine | CRTH2 antagonist (1) | overflow | bronchoprotection |
|---|---|---|---|
| [mg/kg p.o.]⁵⁾ | [mg/kg p.o.]¹⁾ | [ml] | [%] |
| 1.0 | 0.0 | 35 | 42 |
| 1.0 | 0.01 | 29 | 52 |
| 1.0 | 0.03 | 17 | 72 |
| 1.0 | 0.1 | 8 | 87 |
| 1.0 | 0.3 | 9 | 85 |
| 1.0 | 1.0 | 14 | 77 |
| 1.0 | 3.0 | 21 | 65 |
| 1.0 | 10.0 | 26 | 57 |

¹⁾CRTH2 antagonist (1) was administered in the form of its ethylenediamine.salt; the dose is calculated based on the free compound of formula (1);
⁵⁾Cetrizine was administered in the form of its hydrochloride salt; the dose is calculated based on free cetrizine

### C. Conclusion

As can be seen from the results above, CRTH2 antagonist of formula (1) having no effect in this assay by itself induces a significant enhancement of bronchoprotection when being combined with an antihistaminic compound.

1. A pharmaceutical composition comprising a CRTH2 antagonist of formula (1), 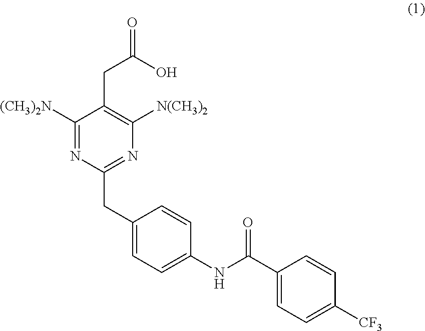
or salts thereof with pharmacologically acceptable acids or bases, and at least one histamine receptor antagonist.

2. The pharmaceutical composition according to claim 1, wherein the CRTH2 antagonist of formula (1) is present as a salt with a pharmacologically acceptable base, wherein the base is an amine selected from primary amines, including methylamine, ethylamine, ethanolamine, tris(hydroxymethyl)aminomethane, and ethylenediamine; secondary amines selected from dimethylamine, diethylamine, diisopropylamine, dibutylamine, di-sec-butylamine, dicyclohexylamine, diethanolamine, meglumine, pyrrolidine, piperidine, piperazine, and benzathine; tertiary amines selected from trimethylamine, triethylamine, triethanolamine, and 1-(2-hydroxyethyl)-pyrrolidine; quaternary ammoniums selected from choline, tetramethylammonium, and tetraethylammonium.

3. The pharmaceutical composition according to claim 2, wherein the amine is selected from ethylenediamine and choline.

4. The pharmaceutical composition according to claim 1, wherein the histamine receptor antagonist is selected from acrivastine, azatadine, azelastine, bamipine, brompheniramine, carbinoxamine, cetirizine, chlorphenoxamine, chlorphenaramine, clemastine, cexchlorpheniramine, cyproheptadine, desloratidine, dexbromphenarimine, dexchlorpheniramine, dimenhydrinate, dimetinden, diphenhydramine, doxylamine, ebastine, emedastine, epinastine, fexofenadine, hydroxyzine, ketotifen, levocetirizine, levocabastine, loratadine, meclozine, methdilazine, mizolastine, olopatadine, phenindamine, pheniramine, phenyltoloxamine, promethazine, pyrilamine, tecastemizole, trimepramine, trimethobenzamide, triprolidine, JNJ-7777120, PF-2988403 and CZC-13788, optionally in racemic form, as enantiomers, diastereomeres or as pharmacologically acceptable salts.

5. The pharmaceutical composition according to claim 4, wherein the histamine receptor antagonist is selected from azelastine, cetirizine, desloratidine, ebastine, epinastine, fexofenadine, hydroxyzine, ketotifen, levocetirizine, loratadine and olopatadine and pyrilamine.

6. The pharmaceutical composition according to claim 5, wherein the histamine receptor antagonist is selected from cetirizine, desloratidine, fexofenadine and levocetirizine.

7. A unit-dosage form comprising the pharmaceutical composition according to claim 1, wherein said pharmaceutical composition in said unit-dosage form comprises the CRTH2 antagonists of formula (1) in an amount of from 1 mg to 1000 mg.

8. The unit-dosage form according to claim 7, wherein said pharmaceutical composition in said unit-dosage form comprises the histamine receptor antagonist in an amount of from 0.1 to 1000 mg.

9. The unit-dosage form according to claim 8, wherein said pharmaceutical composition in said unit-dosage form comprises the CRTH2 antagonist of formula (1) in an amount of from 1 mg to 1000 mg and the histamine receptor antagonist in an amount of from 0.1 to 1000 mg.

10. A method of using a pharmaceutical composition according to claim 1 for treating respiratory and inflammatory diseases and conditions.

11. The method according to claim 10, wherein the respiratory and inflammatory diseases or conditions are selected from asthma, allergic and non-allergic rhinitis, allergic bronchitis, allergic conjunctivitis, farmers disease, nasal polyposis, chronic uritcaria, chronic sinusitis, atopic dermatitis, chronic rhinosinusitis and acute rhinosinusitis.
